(19) United States
(12) Patent Application Publication
(10) Pub. No.: US 2004/0018959 A1
(43) Pub. Date: Jan. 29, 2004

(54) System and methods of lipid removal from the body

(75) Inventors: Randall S. Hickle, Lubbock, TX (US); Vasiliy Smirnov, Lubbock, TX (US)

Correspondence Address: HOGAN & HARTSON LLP, IP GROUP, COLUMBIA SQUARE, 555 THIRTEENTH STREET, N.W., WASHINGTON, DC, 20004, US

(73) Assignee: Randall S. Hickle

(21) Appl. No.: 10/428,005

(22) Filed: May 2, 2003

Related U.S. Application Data
(60) Provisional application No. 60/376,835, filed on May 2, 2002.

(51) Int. Cl. A61K 38/26; C11B 1/00; A61K 31/56

(52) U.S. Cl. 514/2; 514/169; 554/8

(57) ABSTRACT

System and methods for lipid removal from the body, and more particularly, system and methods for treating obesity by lipid removal. In an embodiment, the methods of the present invention include the steps of delivering a hormone activator to the body to initiate the conversion of triglycerides to free fatty acids, and removing the free fatty acids from the body. The invention also includes a system for removing lipids from the body comprising a delivery means for delivering a hormone activator to an adipocyte and a removal means for removing fatty acids from the body.

### CROSS REFERENCE TO RELATED APPLICATIONS

This application claims priority under 35 U.S.C. §119(e) to U.S. patent application Ser. No. 60/376,835 entitled “System and Methods of Lipid Removal From the Body” filed May 2, 2002, which is incorporated herein in its entirety by reference.

### BACKGROUND OF THE INVENTION

1. Field of the Invention

The present invention relates to system and methods for lipid removal from the body, and more particularly, to non-surgical system and methods for treating obesity by lipid removal.

2. Discussion of the Related Art

Obesity results in morbidity and mortality, and is aesthetically undesirable. Clinical observations have suggested a connection between obesity (particularly in its extreme forms) and a variety of illnesses, including hypertension, hypercholesterolemia, diabetes, cancer and coronary artery heart disease. As a result, there are many proposed solutions for this growing problem. Solutions range from suggestions to increase exercise and decrease caloric intake to advice on the use of herbs and aroma-therapy to achieve the desired result, weight reduction. Further, there is a growing use/abuse of medications that increase metabolic rate such as amphetamines, beta agonists, and thyroid stimulating hormones. Some of these illicit and prescription medications are addicting and some have severe potential complications including mortality risks.

Even if the above-mentioned approaches do produce weight loss, it is often observed that excess body fat is removed more rapidly from some parts of the body than from other parts. U.S. Pat. Nos. 4,525,359 and 4,588,724, Greenway, III et al., provide a treatment method for achieving a selective loss in body weight during a weight control program. These patents disclose the selective delivery of an active ingredient that encourages reduction of fatty tissue to a portion of the body having a regional body fat deposit, so that a selective acceleration in reduction of the regional body fat deposit is achieved during the general weight control program. According to these patents, preferred active ingredients that encourage reduction of fatty tissue include beta adrenergic stimulators and alpha-2 adrenergic inhibitors, or combinations thereof. These compounds, often referred to as hormone activators, encourage lipolysis in the fat cell by stimulating an intracellular reaction that results in the conversion of triglycerides to fatty acids. This treatment for selective weight control does not provide a method for fat removal. Moreover, to obtain results, the patient is required to adhere to a general weight loss program (i.e., patients were encouraged to exercise by walking and to adhere to a balanced diet restricted to 1200 calories per day). Even with the assistance of regionally-administered hormonal stimulators of lipolysis, many people who desire weight reduction have difficulty maintaining such a diet and exercise program for a sufficient term to achieve the desired result.

In addition to the aforementioned treatments, many obese patients seek surgical remedies, such as liposuction. In liposuction, a patient is typically subjected to general anesthesia, and needles are used to inject local anesthetic and vasoconstrictive agents (such as epinephrine) into a fat depot. A suction rod is then inserted through the skin into the fat depot, and fat cells (adipocytes) are removed in their entirety along with several other materials and cell types comprising organic structures in the surrounding tissue architecture, such as collagen, fascia, blood vessels, and nerves. The risks of this procedure include bleeding, drug overdose (particularly from excess administration of local anesthetics and adrenergic agents such as epinephrine), and complications from conscious sedation or general anesthesia. Moreover, the removal of fat cells strips the body of an important component of its buffering system to store excess calories. Thus, if weight is regained after liposuction, it must be stored in other sites that have potentially greater risks, such as the lining of the blood vessels, which may cause accelerated arteriosclerosis. Finally, the cosmetic results of liposuction have frequently been less than optimal, including a lumpy appearance and scarring.

### SUMMARY OF THE INVENTION

Accordingly, the present invention is directed to system and methods for removal of lipids from the body that substantially obviates one or more of the problems due to limitations and disadvantages of the conventional methodologies. In an embodiment, the methods of the present invention include the steps of delivering a hormone activator to initiate the conversion of triglycerides to free fatty acids, and removing the free fatty acids. In an alternate embodiment, the invention includes a system for removing lipids comprising a hormone delivery device for delivering a hormone activator to an adipocyte and a fatty acid removal device for removing fatty acids.

Additional features and advantages of the invention will be set forth in the description which follows, and in part will be apparent from the description, or may be learned by practice of the invention. It is to be understood that both the foregoing general description and the following detailed description are exemplary and explanatory and are intended to provide further explanation of the invention as claimed.

### BRIEF DESCRIPTION OF THE DRAWINGS

The invention will be more clearly understood by reference to the following detailed description of exemplary embodiments in conjunction with the accompanying drawings, in which:

FIG. 1 is a flow chart showing the method in accordance with an embodiment of the invention;

FIG. 2 is a diagram showing an embodiment of the system of the present invention;

FIG. 3 is a microscopic image of white adipose tissue;

FIG. 4 shows an embodiment of a peristaltic device according to the present invention;

FIG. 5 depicts the structure of Stearic Acid;

FIG. 6 depicts the structure of Tristearin;

FIG. 7 shows the surface region of a cell and its free energy graph; and

FIG. 8 depicts the structure of Glycocholic Acid.

### DETAILED DESCRIPTION OF A PREFERRED EMBODIMENT

Reference will now be made in detail to embodiments of the present invention, examples of which are illustrated in the accompanying drawings.

The present invention is embodied in a system and methods for removing lipids from the body that is time efficient and safe, involves minimal pain, has a low risk of bleeding, uses natural hormones (e.g., isoproterenol, epinephrine, norepineprine, glucose, insulin, glucagon, ACTH, secretin, vasopressin, glucocorticoids, growth hormone) or their analogues to stimulate lipolysis, involves no surgery (e.g., minimal removal of fat cells, interstitial connective tissues, small blood vessels, and nerves), and produces improved cosmetic results compared to liposuction. The methods of the present invention may be performed periodically and repeatedly in a human to manage total body fat over many years. The potential medical benefits of this obesity treatment include a reduction of high blood pressure, cure for adult onset diabetes, and a reduction in the incidence of degenerative joint disease, sleep apnea, elevated serum triglycerides, and elevated serum cholesterol. Indeed, if serum triglycerides and cholesterol could be reduced while leaving fat cells intact and functioning normally, it is possible that the buffering system of the body for caloric excess could be restored. If this was achieved, then it is possible that disease states associated with atherosclerosis could be reduced or reversed. The potential medical benefits of treating or reducing the progression of atherosclerosis include a decrease in the risk of heart attack and stroke.

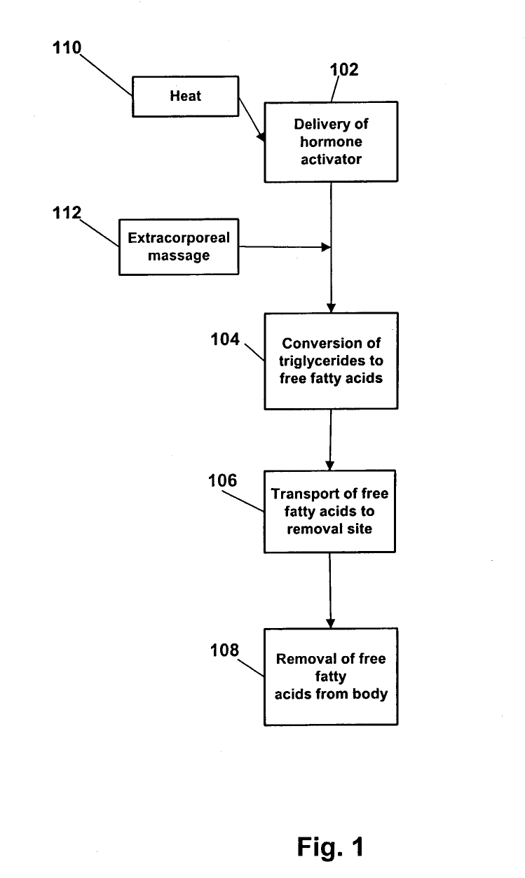

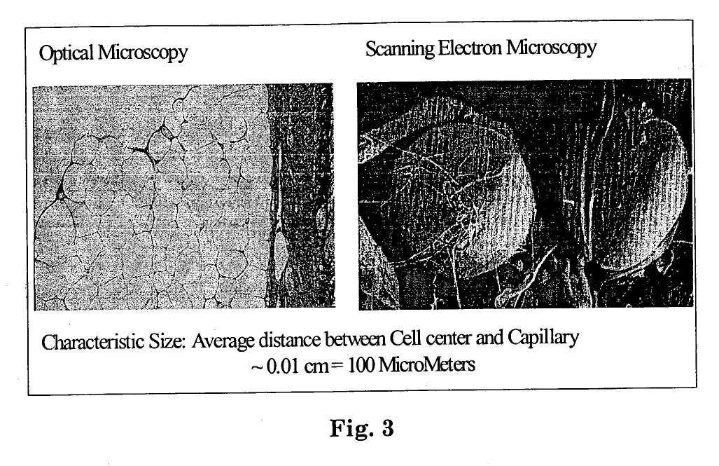

FIG. 1 is a flow chart illustrating a method for lipid removal from the body in accordance with an embodiment of the invention. In FIG. 1, the process begins with step 102. In step 102, a hormone activator is delivered to an adipocyte or fat cell. In an embodiment of the present invention, the hormone activator may be delivered to the external environment in the region of the adipocyte. FIG. 3 shows exemplary microscopic images of adipocytes. Examples of such hormone activators include beta agonists, such as isoproterenol, epinephrine and norepinephrine, and glucagon, which attach to a cell surface receptor on the membrane of the adipocyte and stimulate lipolysis. Other drugs can be added to the hormone activators to increase the magnitude and duration of effect. For example, a class of drugs known as phosphodiesterase inhibitors (e.g., milronine and amrinone) increase the levels of intracellular cyclic AMP produced when beta agonists attach to the cell surface receptor. Cyclic AMP is a critical mediary of lipolysis. In an alternate embodiment, the hormone activators may include steroids, such as glucocorticoids, which go directly inside of the adipocyte to stimulate lipolysis. Other drugs that may be used to cause or enhance the lipolytic effect include glucose or dextrose and insulin.

In accordance with an embodiment of the present invention, the delivery of hormone activators may be effected through several mechanisms. In an embodiment, the method of delivering the hormone activator includes delivery via the blood stream. This is the normal physiological mechanism for delivery of lipolytic activators to adipocytes. An example of this mode of delivery includes the intravenous infusion of epinephrine (e.g., via a solution containing 1 mg of isoproterenol in 250 ml of normal saline infused at a starting rate of 5 ml/hour with subsequent rate adjustments up or down dependent upon the patient's heart rate and blood pressure response). In an alternate embodiment, the hormone activator may be delivered by transcutaneous delivery (i.e., directly across the skin without the use of needles). Examples of transcutaneous delivery include drug delivery, such as scopalamine patches for seasickness, and the delivery of nicotine in nicotine patches.

In a further embodiment, the hormone activator may be delivered directly into the fat tissue by needle insertion. In particular, a needle may be inserted into a fat depot and the hormone activators may be directly delivered into the extracellular space surrounding the adipocytes. An example of such needle delivery includes using a needle, such as a skinny needle (e.g., in the range of 30 gauge), to deliver the hormone activator into the fat tissue under sufficient positive pressure to overcome the elastic recoil of the tissue. If the hormone activators are delivered directly into the fat tissue, dilute concentrations of a local anesthetic (e.g., 0.5% lidocaine) may be simultaneously introduced in the solution to decrease the pain associated with this procedure.

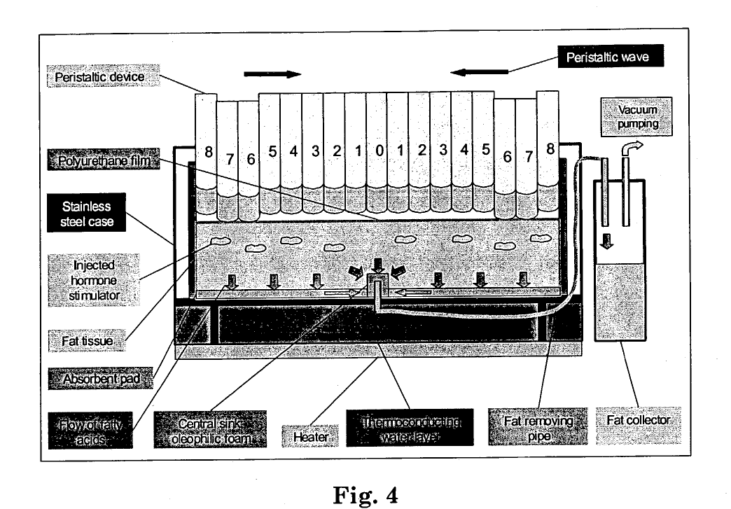

Alternatively, the hormone activator may be delivered by needle insertion at one site in the body, and migrated to other sites by the application of extracorporeal massage (as shown in FIG. 1, step 112). For example, the hormone activator may be injected into the fat tissue surrounding the umbilicus and then migrated radially toward the periphery of the abdomen. An example of a peristaltic device that may be used for this purpose is shown in FIG. 4. Extracorporeal massage is discussed in more detail below, with regard to step 106.

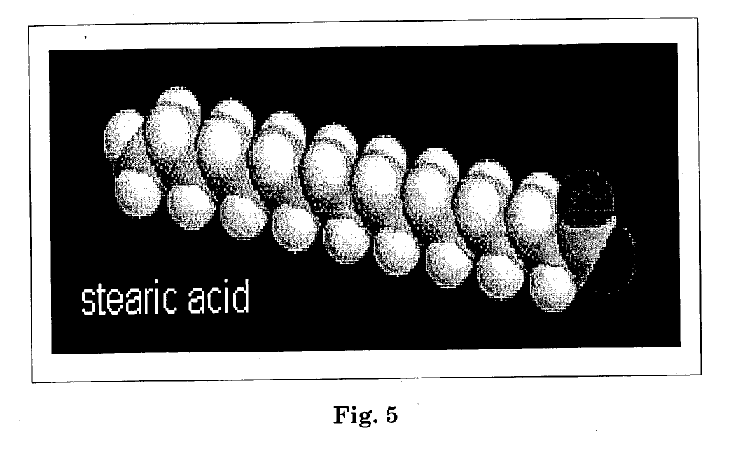

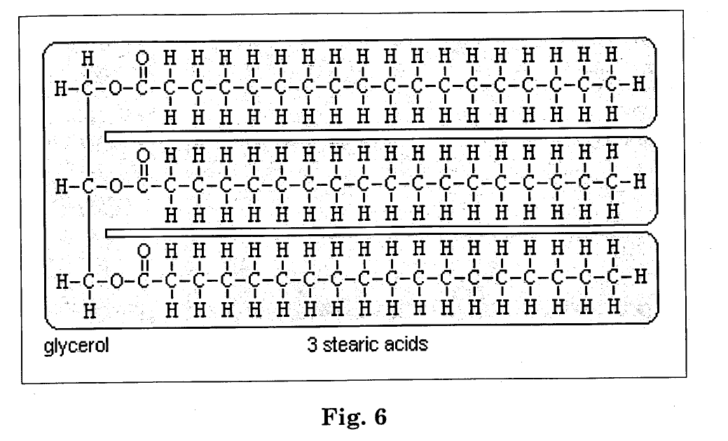

Following delivery of the hormone activator in step 102, the process moves to step 104. In step 104, the delivery of hormone activators to the adipocyte initiates lipolysis resulting in the conversion of triglycerides to free fatty acids. The free fatty acids diffuse through and aggregate on the adipocyte's cell surface. Tristearin, the chemical structure of which is shown in FIG. 6, is an example of a triglyceride that may be converted by the process of the present invention. Stearic acid, the chemical structure of which is shown in FIG. 5, is an example of a free fatty acid that may be formed by such a conversion.

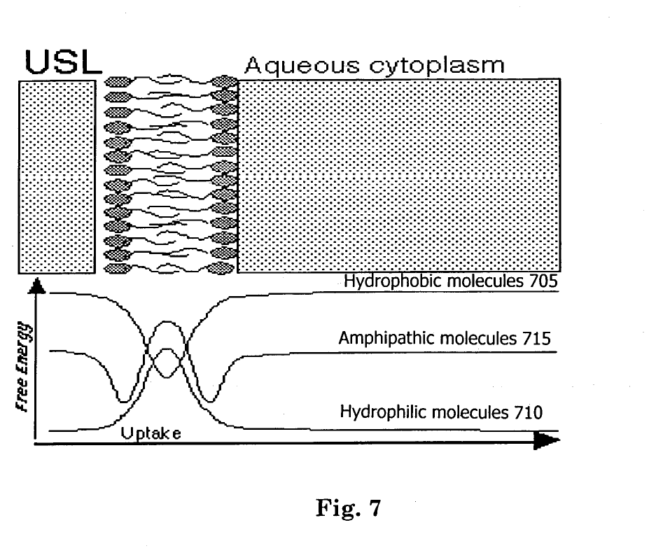

Returning to FIG. 1, the process moves to step 106. In step 106, the free fatty acids may be transported to a removal site. To accomplish this transport, the fatty acids must first leave the adipocyte cell surface. As can be seen in the free energy graph in FIG. 7, hydrophobic molecules 705 (e.g., oils) prefer to be located in the nonpolar middle of the cell membrane where their free energy is at a nadir. Hydrophilic molecules 710 prefer to be located either outside the cell in the aqueous extracellular environment or inside the cell in the aqueous cytoplasm. Amphipathic molecules 715, of which fatty acids are an example, prefer to be located on the inner or outer lining of the cell membrane where their free energy is lowest. Thus, fatty acids have a moderately avid attraction to the adipocyte cell surface and require some assistance in overcoming the energy barrier associated with removal there from.

In an embodiment, the fatty acids may be transported to the removal site via extracellular migration to the removal site. Specifically, in the normal tissue architecture of the fat depot, there is a space (or potential space) between fat cells referred to as the interstitial or extracellular space. The extracellular space in the body is active. For example, it is the site for much of the immunological activity associated with preventing and fighting infectious disease. By injecting fluid such as hormonal activators and/or solubilizing agents, this compartment can be significantly enlarged without adverse consequence.

In normal conditions, there is a large amount of fluid (lymphatic fluid) that flows through the extracellular space. This lymphatic flow is caused by normal bodily motions, particularly by muscle contraction. By applying precisely controlled extracorporeal massage, it is possible to induce the flow of free fatty acids through the extracellular space. Extracorporeal massage is effected by precisely-timed application of “massage fingers” on the surface of the body overlying targeted and lipolytically-activated fat depots. For example, concentric circles of massage fingers could be effected sequentially, resulting in a radius of ever-decreasing size. The fingers could be charged with several pounds of force, resulting in the application of substantial peristaltic force. Thus, the massage fingers create peristaltic wave forces inside the fat tissue that create pressure gradients which cause the directed migration of free fatty acids toward a removal site. An example of massage fingers that may be employed in the present invention includes the numbered (0-8 and 8-0) fingers of the peristaltic device illustrated in FIG. 4.

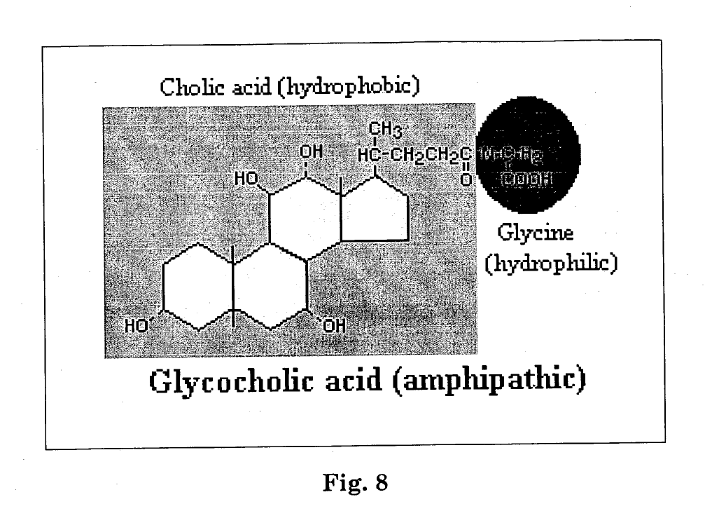

In an embodiment, it is possible to apply extracorporeal massage to cause the free fatty acids to migrate directly through the extracellular space in their original molecular form as an oil. In an alternative embodiment, it is possible to change the free fatty acid state, such as from an oil to a water-soluble blend, with the use of soaps, which aid in the formation of water soluble micelles or other solubilizing compounds such as organic and inorganic solvents. Many soaps have polar (water soluble) and a nonpolar (oil soluble) end. There are naturally-occurring soaps in humans in the form of bile salts. Glycocholic acid, the chemical structure of which is shown in FIG. 8, is an example of a bile acid. The nonpolar end aligns itself toward a source of oil, and the nonpolar end aligns itself toward an aqueous environment. When many soap molecules surround a droplet of oil, oriented with their nonpolar end toward the droplet and their polar end away, they form a structure called a micelle. In this context, micelles are droplets of oil made water soluble by their surrounding layer of oriented soap molecules that have their water soluble ends facing outward. By this mechanism, oil can be solubilized in an aqueous environment. In this application where oil (in the form of fatty acids) is on the surface of adipocytes, the greatest energy change is at the transition point where the oil is released from the cell surface. This energy requirement can be decreased by the formation of micelles.

There are other molecules, in addition to soaps, which may overcome this energy hurdle. One example is albumin. Albumin has a polar exterior surface making it water soluble. The inner surface of an albumin molecule is nonpolar. Instead of promoting the formation of micelles, albumin engulfs a fatty acid. Once the fatty acid is surrounded within the nonpolar inner lining of the albumin molecule, the entire complex is water soluble because of its polar exterior surface.

In a further embodiment, it is possible to introduce enzymes into the extracellular space that catalyze the breakdown of fatty acids into shorter component parts, such as acetic acid. The smaller molecules may be water soluble, thus providing for improved release from the adipocyte surface into an aqueous environment and removal from the body.

When soaps, bile salts, albumin or other solubilizing agents are used, the environment may be manipulated to favor the solubilization of fatty acids. For example, agitation forces, such as those delivered in a washing machine, may help to overcome the energy barrier associated with micelle formation. Similarly, fluid pulses, such as those seen in a dishwashing machine, may help to solubilize fatty acids. Ultrasonic waves may also aid in the process of solubilization. Massage forces delivered by fingers or other suitable means in a kneading or pulsatile, or peristaltic fashion, may also aid in the formation of micelles or other water soluble complexes.

In some patients, well developed fascial planes (e.g., Scarpa's fascia) and scar tissue may prevent the delivery of hormone stimulators and solubilizing agents. In these circumstances, it may be necessary to insert a rod or trocar device through the fat compartment to disrupt the obstacles to fluid flow. The above embodiments of promotion of free fatty acid migration may be implemented individually or in combination with each other or with other means of promoting such migration.

Returning to FIG. 1, once the free fatty acids have been transported to the removal site, the process moves to step 108.

In step 108, the fatty acids may be removed from the body. In an embodiment, the method for removing the free fatty acids from the removal site includes needle extraction. The removal site may be established by insertion of one or more larger gauge needles at a central site that is designated for collection of free fatty acids. The collection needles may generally be 0.5 to 5.0 millimeters in diameter, however it is possible to make them smaller or larger. If the site of insertion has high visibility, a smaller gauge needle (e.g., 20 gauge) may be desired to minimize the cosmetic impact of the insertion site scar. If the site of insertion has minimal visibility (e.g., the side wall of the umbilicus or beneath the folds of the buttocks), however, the collection tube diameter may be significantly increased without adverse consequence. The collection needles may be made of material selected so as to optimize their attraction for lipids, and to maximize molecular forces (such as capillary action) for flow through the needle to the exterior of the body. The collection needles may also be formed of tubes without a “needle” point, similar to intravenous catheters. Such blunt tipped tubes may be inserted with the aid of a needle or other sharp instrument. In an alternate embodiment, suction may be applied to the removal site needles to induce the flow of free fatty acids out of the body. Positive pressure may also be applied over the body to aid in the removal of fatty acids through the needle. The collection needles may have multiple orifices or other means for decreasing the likelihood that they will become plugged if placed under suction. In addition, sucker rods may be inserted into the collection tube to dislodge materials that obstruct their lumen. The orifices of the needles may be designed with support matrices, which minimize the tendency for fatty tissue to obstruct the entrance to the needles. Similarly, removable support matrices may be introduced through the lumen to prevent or remove orifice obstruction. Further, the collection lumen may be rotated on its longitudinal axis or pivoted with the skin entry point being a fulcrum in order to relieve obstructions.

In an embodiment, the removal site may require the insertion of a hollow-lumened tube (e.g., needle or cannula). The preferred removal site depends on the anatomy of the treatment area. For example, if the treatment area includes the umbilicus, it is preferred to insert a removal cannula in the wall of the umbilicus as the resulting scar will have minimal cosmetic consequences. If the treatment area includes the folds of skin at the lower margin of the buttocks, it is preferred to insert a removal cannula in the fold so that any resulting scar will not be easily seen. In an embodiment of the present invention, a hollow-lumened tube (e.g., needle or cannula) may be inserted into the removal site. Negative pressure may be applied to the lumen of the tube to assist in the removal of the fatty acids. A second lumen in the tube may be used to allow the delivery of substances, such as gases or liquids, at the tip of the tube into or near an orifice entering the collection lumen to provide 1) greater solubility of the fatty acids, such as, for example, by the introduction of a soap or other emulsifying agent or solvent, or 2) greater shear force on the inner wall of the removal lumen, such as, for example, by the introduction of air which is removed through the collection lumen at high velocity, or 3) greater dilution of the fatty acids to decrease their viscosity, such as, for example by the introduction of a high volume of a water-based soap solution.

In an alternative embodiment, an oleophilic (oil-loving) fiber, which attracts lipids with capillary forces, may be introduced. In such an embodiment, the oleophilic fibers may be inserted into the fat compartment through the lumen of a catheter or through a small incision where they would take up fatty acids. Once fatty acids have been taken into the fiber, the present invention includes: 1) the removal of the fiber en bloc with the fatty acids, 2) the application of suction to the fiber to remove lipids, or 3) the washing of the fatty acids out of the fiber with the combination of a soap, solvent, or other solubilizing agent applied to the fiber along with suction to remove the lipids there from.

The invention further provides the instillation of a soap, albumin, bile salt, or other solvent or emulsifying agent in the fat compartment to assist in solubilizing the fatty acids to facilitate their removal, followed by the washing out of the fat compartment or adipocyte at the end of the procedure to minimize absorption and systemic effects of the hormone stimulators and solubilizing agent(s).

In a further embodiment of the lipid removal procedure according to the present invention, the albumin-fatty acid complexes may be separated from other blood elements including plasma and blood cells by centrifugal density separation and other means of filtration in a process of extracorporeal circulation.

As shown in FIG. 1, the methods of the present invention further include the optional step 110. In step 110, the temperature of the fat compartment may be regulated at any time during the process of the present invention. The temperature of the fat compartment or adipocyte may be regulated in several ways. For example, heat may be transferred into the fat compartment through the skin. Heat may be applied to the skin via several mechanisms, including conduction, convection, and radiation. Alternatively, the temperature of the fat depots may be regulated by delivering treating solutions, such as hormone stimulators and/or solubilizing agents, at a target temperature (e.g., 40° C.). Temperature management in the targeted tissues may be significant. The core body temperature of humans is typically maintained relatively tightly around an average temperature of 37° C. Most of the fat deposits in the body are in peripheral locations such as in depots over the abdomen and buttocks, however, and the average temperature of these fat deposits is typically in the range 29-35° C. Controlled increases in the temperature of the fat deposits above their normal range may have several important benefits in a lipid removal procedure. These benefits may include an acceleration of intracellular metabolic activities (such as lipolysis), a decreased viscosity of the free fatty acids (resulting in increased flow through the extracellular space), an enhancement of the water solubility of the free fatty acids, and an enhancement of the solubilizing effects of various agents such as soaps and solvents.

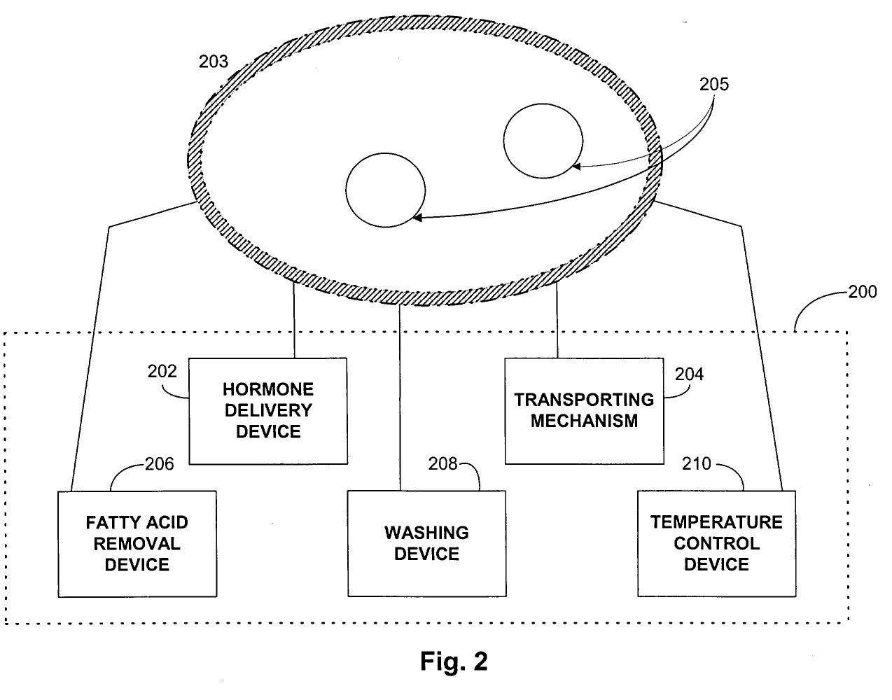

FIG. 2 is a diagram of a system for lipid removal 200 from the body in accordance with an embodiment of the invention. FIG. 2 shows a hormone delivery device 202, a transporting mechanism 204, a fatty acid removal device 206, a washing device 208 and a temperature control device 210, all connectable to the skin surface 203 of a patient. The skin surface 203 includes adipocytes 205.

In operation, the hormone delivery device 202 is used to deliver a hormone activator to the adipocytes 205. Examples of such hormone activators include beta agonists, such as isoproterenol, epinephrine and norepinephrine, and glucagon, which attach to a cell surface receptor on the membrane of the adipocyte and stimulate lipolysis. Other drugs, such as phosphodiesterase inhibitors, can be added to the hormone activators to increase the magnitude and duration of effect. In an alternate embodiment, the hormone activators may include steroids, such as glucacorticoids, which go directly inside of the adipocyte to stimulate lipolysis. Other drugs that may be used to cause or enhance the lipolytic effect include glucose or dextrose and insulin.

The hormone delivery device 202 may deliver the hormone activator to the adipocytes 205 via the blood stream, transcutaneously, or directly into the fat tissue by needle insertion. Dilute concentrations of a local anesthetic (e.g., 0.5% lidocaine) may be simultaneously introduced in the solution to decrease the pain associated with this procedure. Alternatively, the hormone delivery device 202 may deliver the hormone activator by needle insertion at one site in the body, and extracorporeal massage may be used to migrate the hormone activator to a preferred site.

The delivery of the hormone activator to the adipocytes 205 by the hormone delivery device 202 initiates lipolysis, within the adipocytes 205, resulting in the conversion of triglycerides to free fatty acids. Next, the transporting mechanism 204 transports the free fatty acid from the adipocytes 205 to a removal site. In one embodiment, the transporting mechanism 204 induces the free fatty acids to flow through the extracellular space by extracorporeal massage. In an alternative embodiment, it is possible for the transporting mechanism 204 to inject solubilizing agents, such as soaps, albumin and enzymes, which solubilize the fatty acids, for example, by changing the free fatty acid state, such as from an oil to a water-soluble blend.

Once the fatty acids have been transported to the removal site, the fatty acid removal device 206 removes the fatty acids from the body. In an embodiment, the fatty acid removal device 206 removes the fatty acids by needle extraction. The collection needles may have multiple orifices. Suction may be applied to the needles to induce the flow of free fatty acids out of the body. In an alternate embodiment, the fatty acid removal device 206 may remove the free fatty acids by insertion of a hollow-lumened tube. The hollow-lumened tube may include a second lumen to allow delivery of a substance to assist in the removal of the free fatty acids.

Once the free fatty acids have been removed from the body by the fatty acid removal device 206, the washing device 208 may be used to wash out the adipocyte tissue compartment 205 to minimize absorption and systemic affects of the hormone stimulators and solubilizing agent(s).

The temperature control device 210 may be used, at any point during the lipid removal process described in conjunction with the liquid removal system 200, to regulate the temperature of the adipocytes 205.

It will be apparent to those skilled in the art that various modifications and variations can be made in the system and methods for lipid removal of the present invention without departing from the spirit or scope of the invention. Thus, it is intended that the present invention covers such modifications and variations of this invention.

What is claimed is:

1. A method for lipid removal, comprising the steps of:
delivering a hormone activator to an adipocyte to initiate the conversion of triglycerides to free fatty acids; and
removing the free fatty acids.

2. The method of claim 1, wherein the hormone activator includes a beta agonist.

3. The method of claim 1, wherein the hormone activator includes glucagon.

4. The method of claim 1, wherein the hormone activator includes a steroid.

5. The method of claim 1, further comprising the step of adding a drug to the hormone activator to increase the hormone activator's magnitude and duration of effect.

6. The method of claim 5, wherein the drug includes a phosphodiesterase inhibitor.

7. The method of claim 1, wherein the step of delivering the hormone activator includes the step of delivering the hormone activator via the blood stream.

8. The method of claim 1, wherein the step of delivering the hormone activator includes the step of delivering the hormone activator transcutaneously.

9. The method of claim 1, wherein the step of delivering the hormone activator includes the step of delivering the hormone activator to a site by needle insertion.

10. The method of claim 9, wherein the step of delivering the hormone activator to a site by needle insertion further includes the step of adding a local anesthetic to the hormone activator prior to the needle insertion.

11. The method of claim 9, wherein the step of delivering the hormone activator to a site by needle insertion includes delivering the hormone activator to an extracellular space surrounding the adipocyte.

12. The method of claim 9, further comprising the step of applying extracorporeal massage to migrate the hormone activator from the site to other sites.

13. The method of claim 1, further including the step of transporting the free fatty acids from the adipocyte to a removal site.

14. The method of claim 13, wherein the step of transporting the free fatty acids includes inducing the free fatty acids to flow through extracellular space by extracorporeal massage.

15. The method of claim 13, wherein the step of transporting the free fatty acids includes the step of changing the free fatty acids from an oil state to a water-soluble blend state.

16. The method of claim 15, wherein the step of changing the free fatty acids from an oil state to a water-soluble blend state includes using at least one soap to aid in the formation of solubilizing compounds.

17. The method of claim 15, wherein the step of changing the free fatty acids from an oil state to a water-soluble blend state includes using albumin.

18. The method of claim 15, wherein the step of changing the free fatty acids from an oil state to a water-soluble blend state includes using at least one enzyme.

19. The method of claim 1, further including the step of increasing the temperature of the adipocyte.

20. The method of claim 1, wherein the step of removing the free fatty acids includes the step of removing the free fatty acids by needle extraction.

21. The method of claim 20, wherein the step of needle extraction is performed using a needle with multiple orifices.

22. The method of claim 20, wherein the step of needle extraction includes applying suction to the needle.

23. The method of claim 1, wherein the step of removing the free fatty acids further includes the step of removing the free fatty acids by insertion of a hollow-lumened tube.

24. The method of claim 23, wherein the hollow-lumened tube may include a second lumen to allow delivery of a substance to assist in the removal of the free fatty acids.

25. The method of claim 1, wherein the step of removing the free fatty acids further includes the step of washing the adipocyte following the removal of the free fatty acids.

26. A system for removing lipids comprising:
a hormone delivery device for delivering a hormone activator to an adipocyte; and
a fatty acid removal device for removing fatty acids.

27. The system of claim 26, wherein the hormone activator includes a beta agonist.

28. The system of claim 26, wherein the hormone activator includes glucagon.

29. The system of claim 26, wherein the hormone activator includes a steroid.

30. The system of claim 26, wherein the hormone delivery device delivers a drug to the hormone activator to increase the hormone activator's magnitude and duration of effect.

31. The system of claim 30, wherein the drug includes a phosphodiesterase inhibitor.

32. The system of claim 26, wherein the hormone delivery device delivers the hormone activator via the bloodstream.

33. The system of claim 26, wherein the hormone delivery device delivers the hormone activator transcutaneously.

34. The system of claim 26, wherein the hormone delivery device delivers the hormone activator by needle insertion.

35. The system of claim 34, wherein a local anesthetic is added to the hormone activator prior to the needle insertion.

36. The system of claim 34, wherein the hormone delivery device delivers the hormone activator to an extracellular space surrounding the adipocyte.

37. The system of claim 26, wherein extracorporeal massage is used to migrate the hormone activator to a preferred site.

38. The system of claim 26, further comprising a transporting mechanism for transporting the free fatty acids from the adipocyte to a removal site.

39. The system of claim 38, wherein the transporting mechanism induces the free fatty acids to flow through extracellular space by extracorporeal massage.

40. The system of claim 38, wherein the transporting mechanism injects at least one solubilizing agent to change the fatty acids from an oil state to a water-soluble blend state.

41. The system of claim 40, wherein the at least one solubilizing agent includes soaps.

42. The system of claim 40, wherein the at least one solubilizing agent includes albumin.

43. The system of claim 40, wherein the at least one solubilizing agent includes enzymes.

44. The system of claim 26, further comprising a temperature control device for increasing the temperature of the adipocyte.

45. The system of claim 26, wherein the fatty acid removal device removes the free fatty acids by needle extraction.

46. The system of claim 45, wherein the needle extraction includes using a needle with multiple orifices.

47. The system of claim 45, wherein the needle extraction includes applying suction to the needle.

48. The system of claim 26, wherein the fatty acid removal device removes the free fatty acids by insertion of a hollow-lumened tube.

49. The system of claim 48, wherein the hollow-lumened tube includes a second lumen to allow delivery of a substance to assist in the removal of the free fatty acids.

50. The system of claim 26, further comprising a washing device for washing the adipocyte following the removal of the free fatty acids.
